(19) United States
(12) Patent Application Publication
(10) Pub. No.: US 2022/0310269 A1
(43) Pub. Date: Sep. 29, 2022

(54) SYSTEM AND METHOD FOR AIRBORNE VIRAL PATHOGEN DETECTION

(71) Applicant: Joseph Bekanich, Exeter, PA (US)

(72) Inventor: Joseph Bekanich, Exeter, PA (US)

(21) Appl. No.: 17/301,042

(22) Filed: Mar. 23, 2021

(51) Int. Cl. G16H 50/80; A61B 5/00; C12Q 1/70; G01N 33/53; B64C 39/02; H04B 7/185; H04L 29/08; G16H 10/40; G16H 40/67; G06Q 50/26; G01S 19/01

(52) U.S. Cl. CPC G16H 50/80; A61B 5/747; C12Q 1/70; G01N 33/53; B64C 39/024; H04B 7/18506; H04L 67/12; G16H 10/40; G16H 40/67; G06Q 50/26; G01S 19/01; B64C 2201/122

(57) ABSTRACT

The invention is autonomous aircraft disease detection system including an on-board medical device for obtaining medical samples from passengers, flight crew or from the cabin air, a pathogen testing station for analyzing the medical samples, and air samples, and a disease detection docking station using a data processor to produce disease detection results, and a transmitter automatically notifying ground-based facilities of any indication of a viral pathogen.

### CROSS REFERENCE TO RELATED APPLICATIONS

The present application is related to six Provisional Patent Applications entitled as follows: “Autonomous Aircraft Disease Detection and Management System,” filed 23 Mar. 2020 and assigned filing No. 62/993,635; “Autonomous Aircraft. Disease Detection System,” filed 1 Apr. 2020 and assigned filing No. 63/003,862; “Autonomous Infectious Disease Management System,” filed 8 Apr. 2020 and assigned filing No. 63/007,361: and, “Infectious Disease Retention and Transport System,” filed 27 Apr. 2020 and assigned filing No. 63/016,275; and “Autonomous Cryogenic Processing and Transport System” filed Dec. 3, 2020 and assigned file No. 63/120,849; and “Autonomous Cryogenic Processing and Transport. System” filed Dec. 6, 2020 and assigned file No. 63/121,945 all incorporated herein by reference in their entireties.

### FIELD OF THE INVENTION

The present invention relates to a system and method for the field detection of airborne viral pathogens and, more particularly, to a viral pathogen detection system that is adapted to in-flight aircraft usage.

### BACKGROUND OF THE INVENTION

Swab testing kits have been known in the art for some years.

Conventional swab testing kits are subject to contamination from the ambient environment.

### BRIEF SUMMARY OF THE INVENTION

In one aspect of the present invention, a swab module include

Its another aspect of the present invention, a swab testing system includes

In still another aspect of the present invention, a method for obtaining a test sample for virus detection

The additional features and advantage of the disclosed invention is set forth in the detailed description which follows, and will be apparent to those skilled in the art from the description or recognized by practicing the invention as described, together with the claims and appended drawings.

### BRIEF DESCRIPTIONS OF THE DRAWINGS

The foregoing aspects, uses, and advantages of the present invention will be more fully appreciated as the same becomes better understood from the following detailed description of the present invention when viewed in conjunction with the accompanying figures, in which:

FIG. 1 is a diagrammatic illustration of an autonomous aircraft disease detection system as used in an aircraft, in accordance with the present invention;

FIG. 2 is a diagrammatic illustration showing component parts of the autonomous aircraft disease detection system of FIG. 1, with a disease detection docking station functioning as the primary controller when deployed in the aircraft;

FIG. 3 shows a flow diagram illustrating operation of the autonomous aircraft disease detection system of FIG. 1;

FIG. 4 is a diagrammatical illustration of a collection swab module showing a collection swab in a collection swab transport tube, in accordance with the present invention;

FIG. 5 is a view of the collection swab module of FIG. 4 in an opened configuration showing a tubular grip and a tubular chamber;

FIG. 6 is a view of the tubular chamber of FIG. 5 showing an opened chamber cap;

FIG. 7 is a view of the tubular chamber of FIG. 5 showing a swab applicator shaft passing through an annular elastic seal in a chamber wall;

FIG. 8 is a diagrammatical illustration of a collection swab module having a resilient band connecting a tubular grip to a tubular chamber, in accordance with the present invention;

FIG. 9 is a diagrammatical illustration of a face shield with a covered swab port; in accordance with the present invention;

FIG. 10 is a view of the swab part of FIG. 9 in an uncovered state;

FIG. 11 is a diagrammatical illustration of the tubular chamber of FIG. 5 positioned for insertion into the uncovered swab port of FIG. 10;

FIG. 12 shows the collection swab of FIG. 4 or FIG. 8 with the tubular grip removed;

FIG. 13 shows the swab of FIG. 12 with an end cap opened;

FIG. 14 shows the swab of FIG. 13 with the end cap repositioned along the tubular chamber;

FIG. 15 is a diagrammatical illustration of the tubular chamber of FIG. 9 with the chamber cap opened for insertion into the uncovered swab port of FIG. 10;

FIG. 16 is a diagrammatical illustration of the swab of FIG. 15 inserted into the face shield of FIG. 10;

FIG. 17 is a diagrammatical illustration of the tubular chamber of FIG. 10 mated with the swab port on the tubular chamber;

FIG. 18 is diagrammatical illustration of the tubular chamber of FIG. 11 positioning the enclosed swab tor obtaining a sample from the wearer of the face shield;

FIG. 19 is a front view showing the swab inserted into the nasal cavity of the wearer of the face shield;

FIG. 20 is a diagrammatical illustration showing the swab withdrawn from the nasal cavity with the cap positioned for closure;

FIG. 21 is a diagrammatical illustration showing the tubular chamber removed from the swab port and the chamber cap positioned for closure;

FIG. 22 a diagrammatical illustration showing the closed tubular chamber of FIG. 21;

FIG. 23 is a diagrammatical view of the tubular chamber of FIG. 22, showing a removable pull tab feature of the cap anchor collar;

FIG. 24 is an isometric diagrammatical view of a pathogen test station with a testing basin for receiving the tubular chamber of FIG. 23, in accordance with the present invention;

FIG. 25 is a detail view of the testing basin of FIG. 24;

FIG. 26 is a diagrammatical illustration showing the insertion of the tubular chamber of FIG. 20 into the testing basin of FIG. 25;

FIG. 27 is a diagrammatical illustration showing placement of the testing basin of FIG. 25 onto the pathogen test station of FIG. 24;

FIG. 28 is a diagrammatical illustration showing the testing basin of FIG. 25 installed in the pathogen test station of FIG. 24;

FIG. 29 is a diagrammatic illustration showing a swab module for use in the sampling the air in the exhaust ducting, of the aircraft, in accordance with the present invention;

FIG. 30 is a diagrammatic illustration of a gallery panel showing access to an exhaust duct swab port;

FIG. 31 is a detail view of the exhaust duct swab port of FIG. 30;

FIG. 32 is a diagrammatical illustration of the collection swab module of FIG. 4 configured for use with the exhaust duct port of FIG. 30;

FIG. 33 is a diagrammatical illustration of a fluid receptacle including a tapered cylinder with an attached receptacle cover;

FIG. 34 is a detail view of the fluid receptacle of FIG. 34 showing graduation markings;

FIG. 35 is an illustration of a base cover for the fluid receptacle of FIG. 34;

FIG. 36 is a diagrammatical illustration of a fluid flow knob used on the fluid receptacle of FIG. 34;

FIG. 37 is a diagrammatical illustration showing attachment of the fluid reservoir of FIG. 22 to the fluid receptacle of FIG. 34;

FIG. 38 is an illustration showing placement of the fluid reservoir and the fluid receptacle of FIG. 34 into the pathogen detection station of FIG. 21;

FIG. 39 is an illustration of the fluid flow knob of FIG. 36 in a rotated position;

FIG. 40 is an illustration showing placement of an alternate fluid receptacle into the pathogen detection station of FIG. 21;

FIG. 41 is a diagrammatical illustration of a puncture stopper in the fluid receptacle of FIG. 40;

FIG. 42 is a diagrammatical illustration of an air/breath collector including an air tube on a collector base;

FIG. 43 is an exploded view of the air/breath collector of FIG. 42;

FIG. 44 is a top view of the collector base of FIG. 42;

FIG. 45 is a view of microchannels on the top surface of the collector ba of FIG. 44;

FIG. 46 is a cross sectional view of the collector base of FIG. 44;

FIG. 47 is a diagrammatical illustration of an ambient air swab module;

FIG. 48 is a back view of the ambient air swab module of FIG. 47; and

FIG. 49 is an exploded view of the ambient air swab module of FIG. 47.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention.

The present invention relates generally to providing a high standard of reliability in the process of airborne viral pathogen detection in a subject. The disclosed system for the field detection of airborne viral pathogens operates using specimens collected from the ambient environment and/or from available individuals, and has particular application to viral pathogen detection on board an in-flight aircraft.

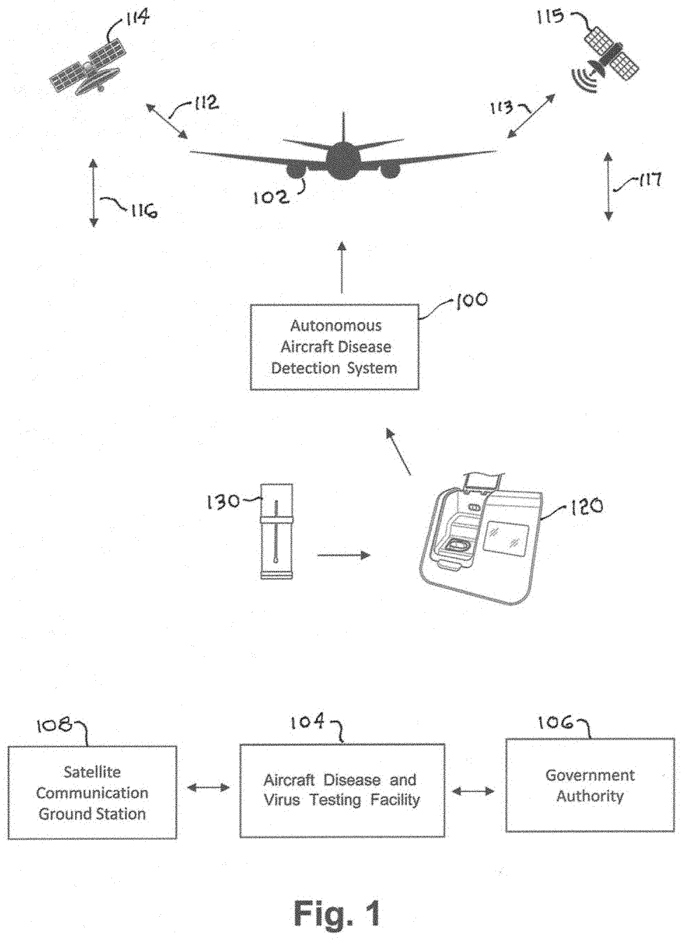

FIG. 1 illustrates an exemplary application of an autonomous aircraft disease detection system 100 as may be incorporated in an aircraft 102, or in other commercial transportation vehicle (not shown), or even in a stationary location (not shown). In an embodiment, a pathogen detection station 120 and a collection swab may include any one of: (i) substrate, (ii) cassette, (iii) custom swab or specimen retention holder, (iv) antigen test cassette, (v) air or breath sampling or retention vessel, (vi) and/or a pathogen/drug test strip/cassette/assay, (vii) condenser, module 130 are exemplary component parts of the autonomous aircraft disease detection system 100, and function as described below. The autonomous aircraft disease detection system 100 operates to continually test, monitor, and detect the possible presence of an emergency medical situation, an airborne viral pathogen, or a disease condition on board the aircraft 102. If such a medical emergency is detected, the autonomous aircraft disease detection system 100 functions to notify appropriate authorities such as, for example, an aircraft disease and virus testing facility 104 and/or a flight crew and/or government authority 106 via a satellite communication ground station 108. It can be appreciated that, because air travel typically transports passengers between thousands of destinations in hundreds of countries, providing the autonomous aircraft disease detection system 100 for use by the airline industry presents a unique method of detecting and mitigating the possible global spread of airborne viral pathogens.

The government authority 106 may include any one of: (i) the Federal Bureau of Investigation (FBI), (ii) the Central Intelligence Agency (CIA), (iii) the Federal Aviation Authority (FAA), (iv) the Federal Emergency Management Association (FEMA), (v) the Office of Homeland Security, (vi) Center for Disease Control. (vii) National Security Administration (NSA), (viii) Transportation Safety Administration (TSA), North American Aerospace Defense Command (NAADC), (ix) local police and safety personnel, and (x) local ‘911-emergency’ response personnel. The monitored emergency disease testing data, health emergency, viral pathogen detection, or technical problem which is identified will have normal, escalated and distress state notifications in its communication data.

As shown in the illustration, and in response to an emergency situation, the aircraft 102 has established a first satellite communication link 112 with a first satellite 114, and the first satellite 114 has established a first ground communication link 116 with the satellite communication ground station 108. In addition, or in the alternative, the aircraft 102 has established a second satellite communication link 113 with a second satellite 115, and the second satellite 15 has established a second ground communication link 117 directly with the government authority or onboard flight crew member 106. Either or both the satellite communication ground station 108 and the government authority 106 may be in data communication with the aircraft disease and virus testing facility 104 while the aircraft 102 is in transit.

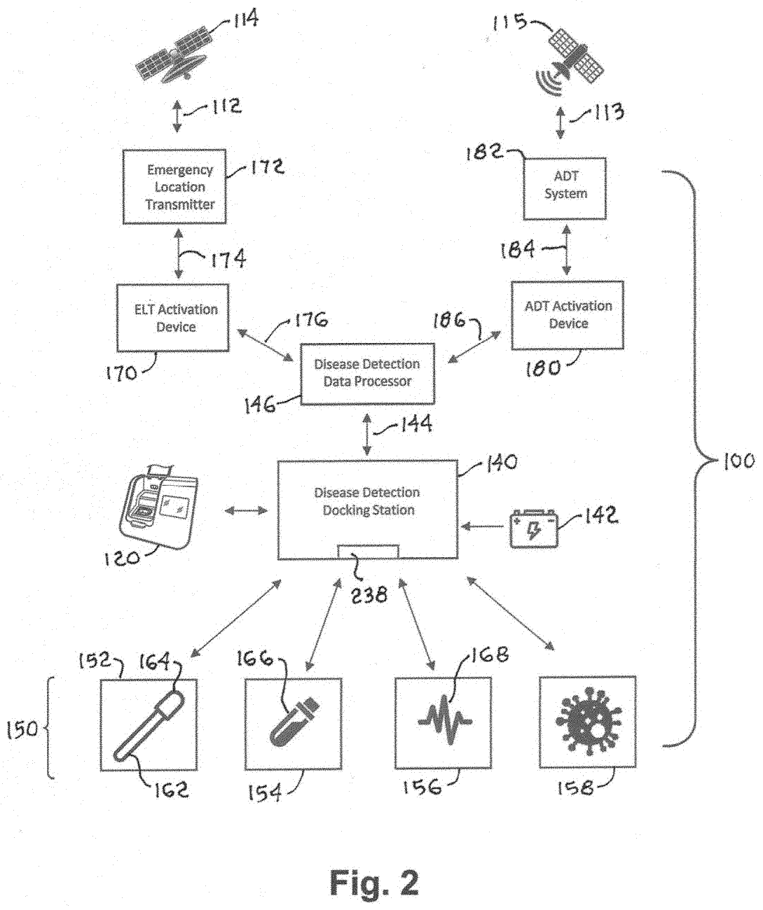

FIG. 2 shows the major components of the autonomous aircraft disease detection system 100. A disease detection docking station 140 functions as the primary controller of the autonomous aircraft disease detection system 100. A power source 142 serves as a backup supply of electrical power so as to make the autonomous disease detection system an autonomous system. Input disease data is provided by a plurality of disease collection samples 150 such as, for example, a physical specimen module 152, a collection sample module 154, an instrument optical reader/reading software/storage device 156, and a particulate matter cassette 158. The input disease data is obtained by mating a selected sample to a disease sample port 148 in the disease detection docking station 140.

The disease detection docking station 140 inputs data and readings from one or more of the disease collection samples 150 and from pathogen test specimens analyzed by the pathogen detection station 120. The disease detection docking station 140 outputs disease detection results to a disease detection data processor 146 via a disease detection data link 144. In accordance with specified protocols, onboard disease status reports are prepared by the disease detection data processor/software 146 for encryption, and transmittal along over an onboard communication network data, which may consists of a third transmitter may be connected to the aircraft wireless network to onboard emergency notification devices over a wireless network software, which may activates; smoke detectors alarm, emergency location transmitter, aural alarm, silent visual alarm to notify authorities or flight crew of a health emergency as well as transmission to one or both of the first. satellite 114 via the first satellite communication link 112, and the second satellite 115, via the second satellite communication link 113.

This aircraft tracking system includes a remote-control emergency activation system. The satellite communication ground station 108 includes tracking software, and video displays used by ground personnel may show the aircraft being tracked with special coding, indicator, aircraft “N” number identifier and or color to identify the aircraft status. The autonomous aircraft disease detection system 100, when thus activated, can concurrently provide real-time tracking information as well as emergency disease management information to at least one government authority. It should be noted that the system and methods described herein are not limited to aircraft but can be applied to any mode of transport, such as public transportation. Alternatively, the system and methods can be advantageously used at stationary locations, such as homes, schools, hospitals, commercial establishments, and social gathering facilities, for example.

The physical specimen module 152 may contain physical material collected by a collection swab 162 tipped with a synthetic material 164, for example, where the collection swab 162 and the synthetic material 164 are exposed to an ambient air flow to collect the sample material. The collection sample module 154 may accommodate a nasal, breath, saliva, saline or mouth rinse, or blood sample 166, for example. The instrument reading storage device 156 can be a solid-state storage fob displaying heart monitoring waveform data 168, for example. The particulate matter cassette 158 may contain samples of a virus or other pathogen filtered from ambient air, for example, using an air filtration/detection device and/or a hydro-chromic indicator and/or reagents, as described in greater detail below. As is known in the relevant art, air detection can be performed by means of a fan source located in a HEPA (High Efficiency Particulate Air) base of an air filtration/detection device, as described in greater detail below, where the fan source is used in conjunction with a shaped panel acting as an air collector or an air deflector, the air being deflected into a channel. The ambient air thus obtained is transmitted through the channel so as to pass across a particulate collector or across one or more collection swabs 162. In an exemplary embodiment, the particulate material thus collected can be used in the pathogen detection station 170, as described above, or in the physical specimen module 242.

The data obtained from one or more of the disease collection samples 150 are analyzed, reformatted, and processed by the disease detection data processor 146 into computer-readable instructions and digital medical data. It may be determined by the autonomous aircraft disease detection system 100 that at least one of a health emergency, abnormal test results indicating presence of viral pathogens from the emergency disease detection data, or a technical problem, is identified in monitored emergency disease detection data on the aircraft 102. Such a situation can occur during a widespread pandemic, such as has been experienced with the SARS-CoV-2 virus and the Wuhan CoVID-19 virus.

If this is the case, the disease detection data processor 146 may send a first activation signal and the processed data to a SATCOM emergency location transmitter (SATCOM/ELT) activation device 170 over a SATCOM/ELT transmitter activation signal link 176. A second activation signal and the digital medical data may similarly be sent to an autonomous distress tracking (ADT) activation device 180 over an ADT transmitter activation signal link 186. Aircraft communication includes, but is not limited to: SATCOM, ACARS, and ADS-B tracking with communication disease detection data processing. Additional testing facilities complementing the autonomous aircraft disease detection system 100 may be located on the ground and remotely linked to the aircraft 102 (not shown) while the aircraft 102 is in transit.

All of the medical and air quality status information used for tracking emergency disease information described above can be autonomously sent from an aircraft, or a mass transit vehicle, or a stationary location, to ground stations for analysis. For example, with additional reference to FIG. 1, it should be understood that the satellite communication ground station 108 is directly linked to the autonomous aircraft disease detection system 100 in the aircraft 102 to track, report and control elements of the autonomous aircraft disease detection system 100 and the aircraft 102. Also, the aircraft disease and virus tracking facility 104 and the government authority 106 may be configured to remotely activate and control any and all information produced by the disease detection data processor 146 via the satellite communication ground station 108.

In response to the first activation signal, the SATCOM/ELT activation device 170 activates an emergency location transmitter 172 via a SATCOM/ELT data link 174. In response to the second activation signal, the ADT activation device 180 activates an autonomous distress tracking system 182 via an ADT data link 184. The aircraft emergency location transmitter 172 and the autonomous distress tracking system 182 conform to emergency location transmitter or emergency location beacon standards defined by the International Civil Aviation Organization (ICAO) for Global Aeronautical Distress Safety System (GADSS), International Maritime Organization (IMO) for Global Maritime Distress Safety System (GMDSS), 911 Services, or Emergency Services functioning to broadcast distinctive signals and data on designated frequencies.

With additional reference to FIG. 1, the computer-readable instructions and digital medical data processed by the disease detection data processor 146 is transmitted to the satellite communication ground station 108, to the government authority 106, and to the aircraft disease detection and virus testing facility 104 via the first satellite 114 and the first satellite communication link 116, and via the second satellite 115 and the second satellite communication link 117. The computer-readable instructions and digital medical data may also be encrypted in secure storage on or off the aircraft 102.

A health emergency, or abnormal test results indicating the presence of viral pathogens, will automatically trigger real-time tracking of the aircraft 102 via either or both the first satellite 114 and the second satellite 115. The geographical position of the aircraft 102 can be determined and monitored by acquiring signals transmitted by three or more Global Positioning System (GPS) satellites via a GPS communication link, as is well-known in the art. In an exemplary embodiment, onboard disease data can be transmitted, along with the current GPS location of the aircraft 102, to continually track the migration of a possibly infectious onboard disease to the location of the next landing destination of the aircraft 102. Such data can be collected from most or all the airborne flights over a particular land mass to track in real time, and provide early warning of, the possible spread of an infectious disease.

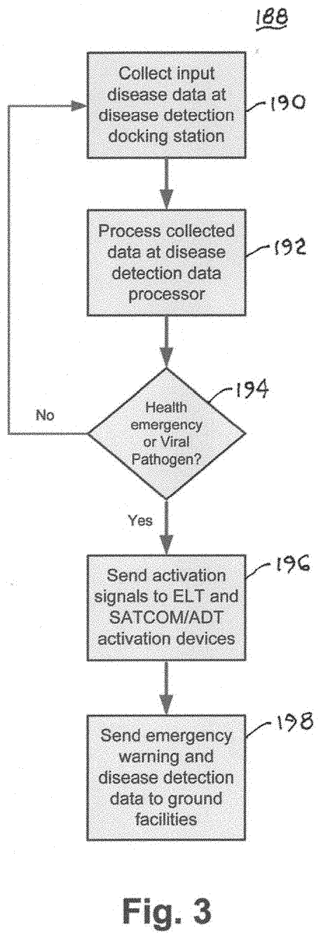

FIG. 3 is a flow chart 188 illustrating, an exemplary operation of the autonomous aircraft disease detection system 100. At step 190, one or more of the disease collection samples 150 are inserted into the disease sample port 148 whereby the pertinent environmental and disease data can be, obtained. Alternatively, the pathogen detection station 120 can be used to obtain disease data or detect the presence of viral pathogens for analysis by the disease detection docking station 140. The relevant physical specimens, collection samples, instrument readings, and particulate matter can be analyzed by the disease detection docking station 140. The environmental and disease data input is processed with the disease detection data processor 146, at step 192. If, at decision block 194, a health emergency or abnormal air quality result or a viral pathogen is not found, the process returns to step 190.

If, at decision block 194, a health emergency or abnormal air quality is determined or a viral pathogen is detected, activation signals are sent to the ELT activation device 170 and/or to the ADT activation device 180, at step 196. The aircraft emergency location transmitter 172 and the autonomous distress tracking system 182 transmit emergency messages and the disease testing data to the satellite communication ground station 108, to the government authority 106, and/or to the aircraft disease and virus testing facility 104, at step 198.

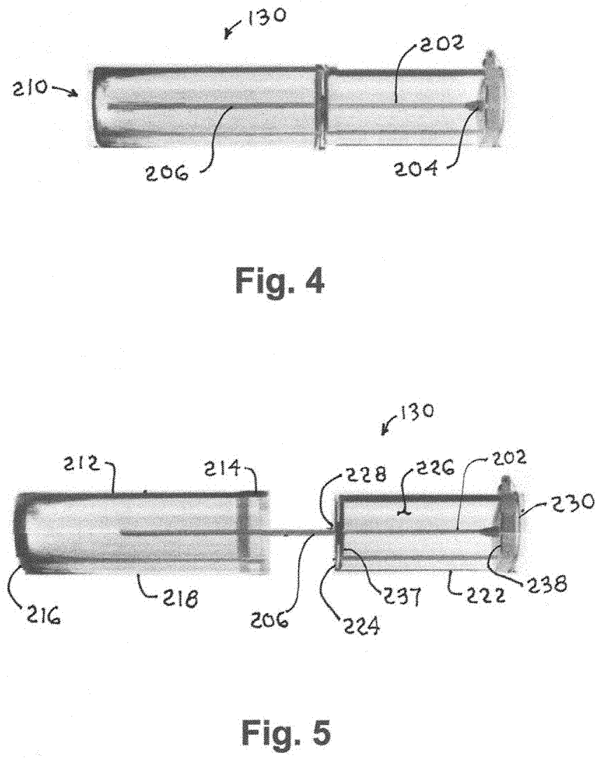

FIG. 4 shows the collection swab module 130 in greater detail. The collection swab module 130 includes a collection swab 202 having a swab tip 204 on a swab applicator shaft 206, configured for use in obtaining a specimen from a test subject. The swab applicator shaft 206 is preferably a slender, solid or hollow cylinder, manufactured from a flexible, inert material, such as a plastic. The swab tip 204 may be a matrix formed from a synthetic fiber such as, for example, polyester, nylon or foam. The ends of the swab fibers may be frayed, or otherwise treated, to increase the surface area available for specimen collection.

The collection swab 202 is enclosed within a swab transport tube 210. The swab transport tube 210 is constructed and configured to protect the swab tip 204, in particular, from ambient environmental contamination, when in storage, when used to obtain a biological sample from the test subject, and when the collection swab 202 is being transported from the test subject to an analysis device (not shown). The swab transport tube 210 is further designed to protect the swab applicator rod 206 from damage, when in storage and during transportation.

The swab transport tube 210 includes a tubular grip 212 and a tubular chamber 222, as seen in FIG. 5. In the particular configuration shown, the tubular grip 212 is a substantially cylindrical enclosure 218 with an oval or a circular cross section. The cylindrical enclosure 218 includes a grip collar 214 at a first grip end and an enclosure base 216 at a distal second grip end. The grip collar 214 is a wider section of the cylindrical enclosure 218 in that the inside opening of the grip collar 214 opening is dimensionally the same as the outside surface of the tubular chamber 222. This configuration provides for a press fit, or friction fit, between the grip collar 214 and the tubular chamber 222.

The tubular chamber 222 is thus frictionally secured to the tubular grip 212 when the collection swab 202 is being stored, freeze-dried, cryogenically stored and or transported. The grip collar 214 also prevents the tubular chamber 222 from unrestricted sliding into the tubular grip 212 and possibly damaging the swab applicator shaft 206. The enclosure base 216 provides a closed end for the tubular grip 212, the collection swab module 130 thus forming an airtight volume. In an alternative exemplary embodiment (not shown), the tubular grip can be configured as a substantially conical component, which may be fluid absorbency that may also store and extract fluid by certain methodologies, which may have a barrier layer for initial non-absorption until it becomes saturated with fluid and is not limited to fluid but may absorb breath, air, gas or the like; with the outer surface of the conical component tapering from a grip collar of specified width to a smaller rounded closed end.

It should be understood that the purposes of the tubular grip 212 are to: (i) provide protection for the swab applicator shaft 206, (ii) provide a closed volume when fitted over the tubular chamber 222, and (iii) serve as a comfortable grip for a user. Accordingly, the present invention is not limited to the linear design shown as the tubular grip may be fabricated in other configurations, such as, for example, a curved tubular component, a tubular component having a smoothly varying outside surface dimension, a tubular component with a circular cross section, or a tubular component having at least one flat surface region on a circumference to prevent the collection swab module 130 from rolling when placed on a surface.

In an exemplary embodiment, the tubular chamber 222 is a hollow cylindrical component having a chamber seal 224 secured to a first chamber end 237, and a chamber cap 230 covering a second chamber end 238. The chamber seal 224 includes a substantially circular opening retaining an annular elastic grommet 228, made of flexible material commonly used in medical equipment, sized and shaped to allow the swab applicator shaft 206 to be passed through the chamber seal 224, but preventing ambient air/fluid from entering the tubular chamber 222. In the configuration shown, the annular elastic grommet 228 is positioned substantially in the center of the chamber seal 224. The swab tip 204 is thus disposed in a sealed chamber volume formed by the tubular chamber 222, the chamber seal 224, and the chamber cap 230, with the swab applicator shaft 206 extending into the tubular grip 212.

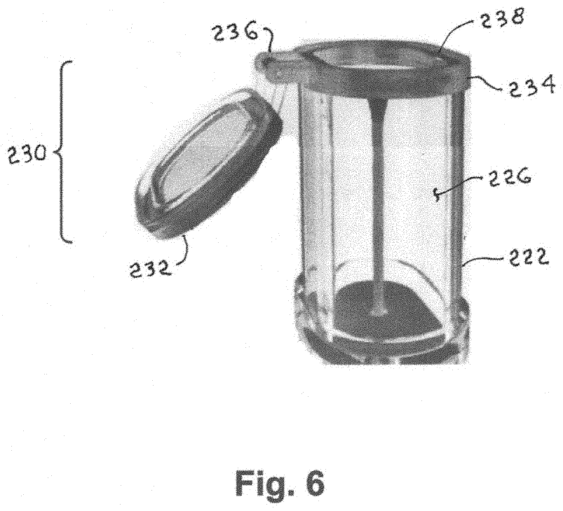

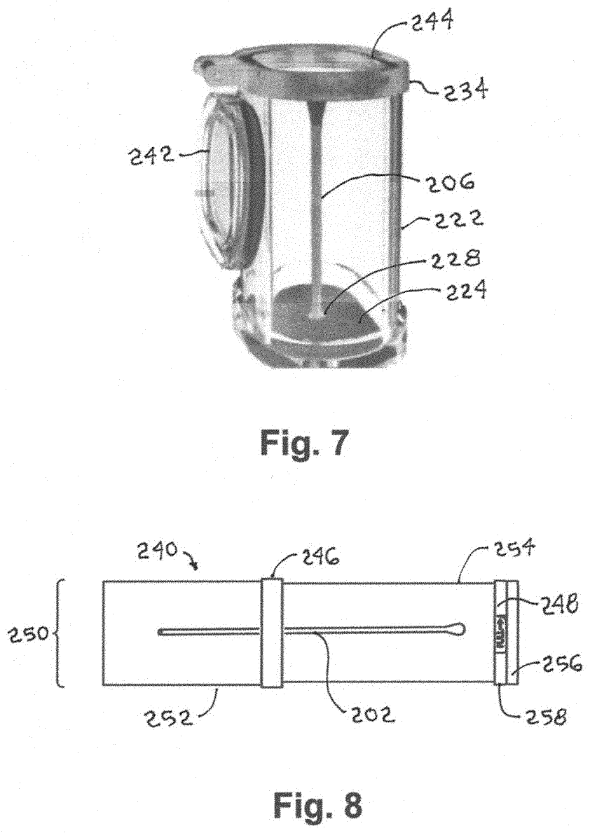

The chamber cap 230 includes a chamber cover 232 attached with a cap hinge 236 to a cap anchor band 234, as shown in FIGS. 6 and 7. The cap anchor band 234 is normally positioned at the second chamber end 238, but can be moved along the outer chamber surface 226, as explained in greater detail below. The chamber cover 232 includes a raised cover ridge 242 sized and shaped to physically interlock with a corresponding recessed channel 244 in the cap anchor band 234. In the exemplary embodiment shown, the raised cover ridge 242 and the recessed channel 244 are substantially oval in shape and are mirror images of one another. It should be understood that other geometric shapes, including asymmetrical shapes, can be used for the ridge/channel pair as best suited for a particular application. Insertion of the raised cover ridge 242 into the recessed channel 244 requires the application of user force to produce a ‘snapping’ action so as to lock the chamber cover 232 into place onto the cap anchor band 234. The chamber cover 232 is thus configured to allow only intentional opening or closing of the tubular chamber 222 by a user.

Also shown in FIG. 7 is the annular elastic grommet 228 as retained in the chamber seal 224. The swab applicator rod 106 passes through the annular elastic grommet 128 which thus functions to retain the swab applicator rod 106 in position, as shown, while allowing the swab applicator rod 106 to be moved longitudinally within the tubular chamber 122. Preferably, the annular elastic grommet 128 is formed from a compliant material, such as plastic or a composite. The annular elastic grommet 128 also allows the user to tilt the swab applicator rod 106 from side to side without admitting air into the tubular chamber 122, as described in greater detail below.

In an exemplary embodiment, an alternative collection swab module 240 includes a swab transport tube 250 housing the collection swab 202, shown in FIG. 8. The swab transport tube 250 includes a tubular grip 252 with a resilient band 246 used to frictionally hold a tubular chamber 254 to the tubular grip 262. Note that the resilient band 246 serves a function similar to the grip collar 214 on the tubular grip 212 (not shown). The tubular chamber 254 includes a chamber cap 256 with a pull tab 248 forming a portion of a cap anchor band 258. By breaking the pull tab 248 away from the cap anchor band 258, a user is able to remove the chamber cap 256 from the tubular chamber 254.

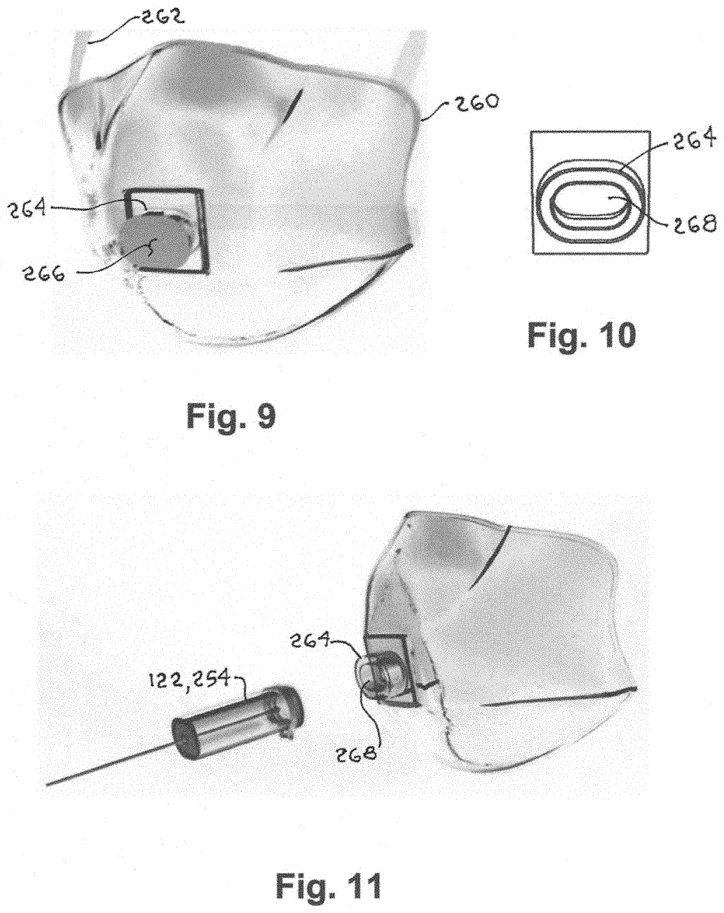

There is shown in FIG. 9 a plastic face shield 260 configured to fit against the face of the test subject being examined for the presence of a viral pathogen or other airborne substance. Adjustable or elastic straps 262 may be used to hold the plastic face shield 260 in place so as to provide access to the nose and/or the mouth of the test subject. A peel-away covering 266 can be removed to expose an access opening 268 in a swab access port 264, shown in greater detail in FIG. 10. The swab access port 264 is configured to mate with both the uncapped ends of the tubular chamber 222 and with the tubular chamber 254, as shown in FIG. 11.

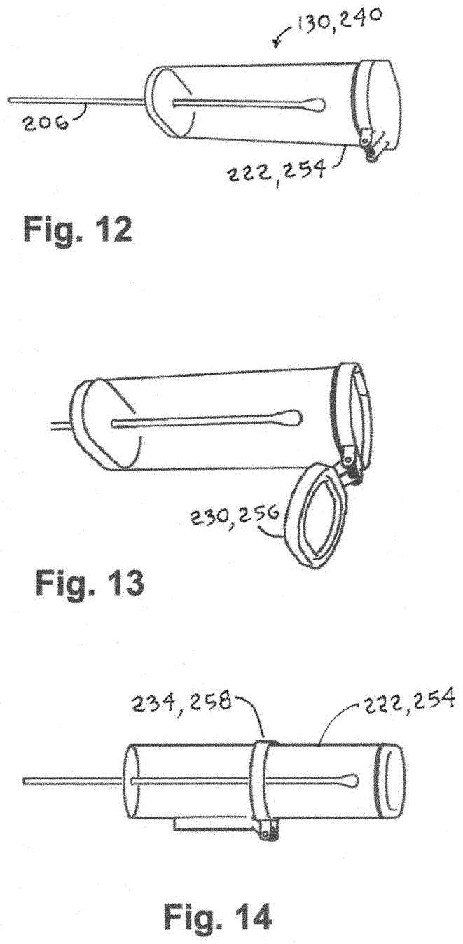

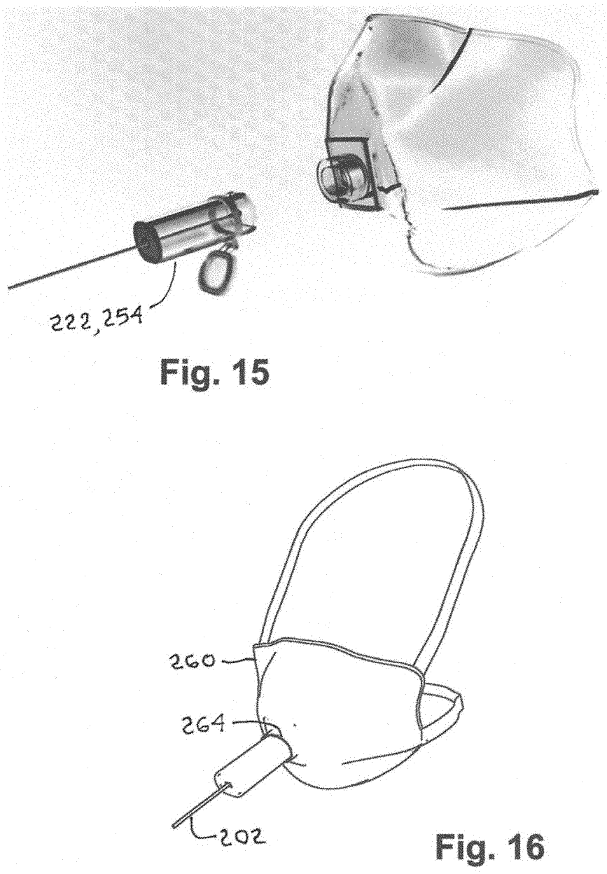

FIGS. 12-14 show the sequence followed by a user in preparing the collection swab nodule 130, 240 for mating with the swab port access port 264 on the plastic face shield 260. The tubular grip 212, 252 is removed from the respective tubular chamber 222, 254 to expose the swab applicator rod 206, in FIG. 12. The chamber cap 230, 256 is opened, in FIG. 13. The chamber anchor band 234, 258 is repositioned on the tubular chamber 222, 254, in FIG. 14. The tubular grip 212, 122 (not shown) has been removed from the respective tubular chamber 222, 254, as shown in FIG. 15. The user can insert the collection swab 202 into the swab access port 264 on the plastic face shield 260, as shown in the simplified illustration of FIG. 16.

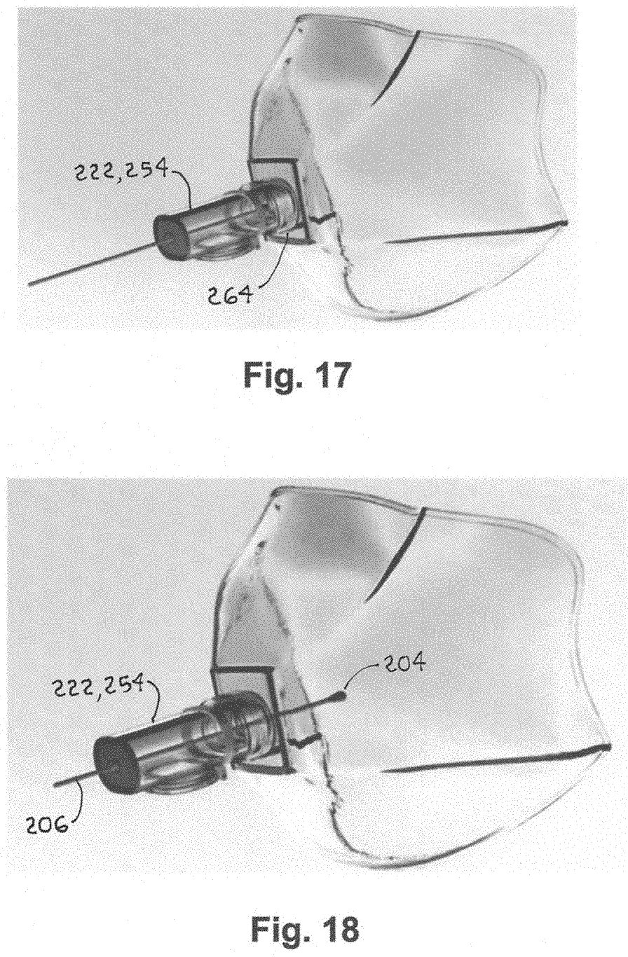

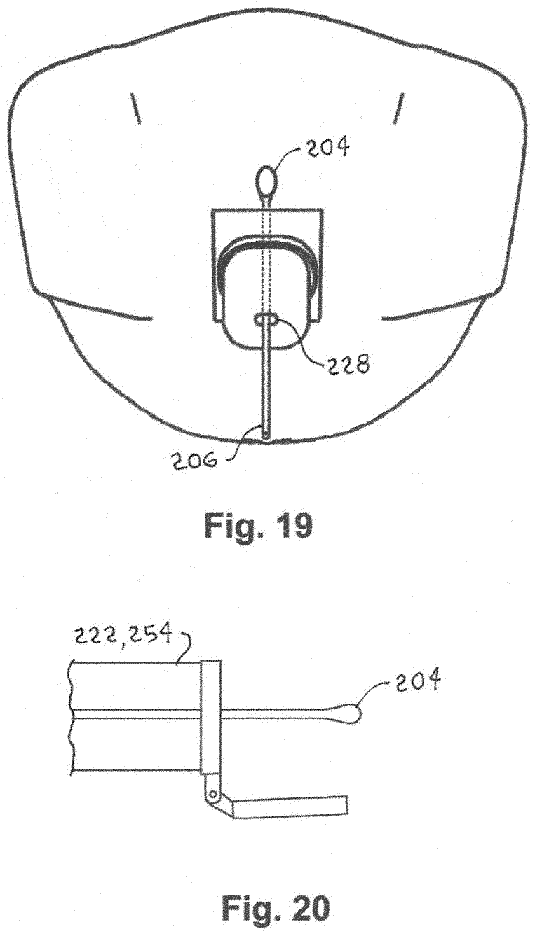

After the tubular chamber 222, 254 has been inserted into the swab port 264, as shown in FIG. 17, the user holds the tubular chamber 222, 254 and, using the swab applicator shaft 206, positions the swab tip 204 in the nostril of the test subject for a nasopharyngeal test, or in the mouth of the subject, to obtain a fluid sample, as shown in FIG. 18. FIG. 19 is a simplified diagrammatical view showing how the swab applicator shaft 206 is retained in the annular elastic grommet 228 while the user moves around the swab tip 204 to obtain the sample.

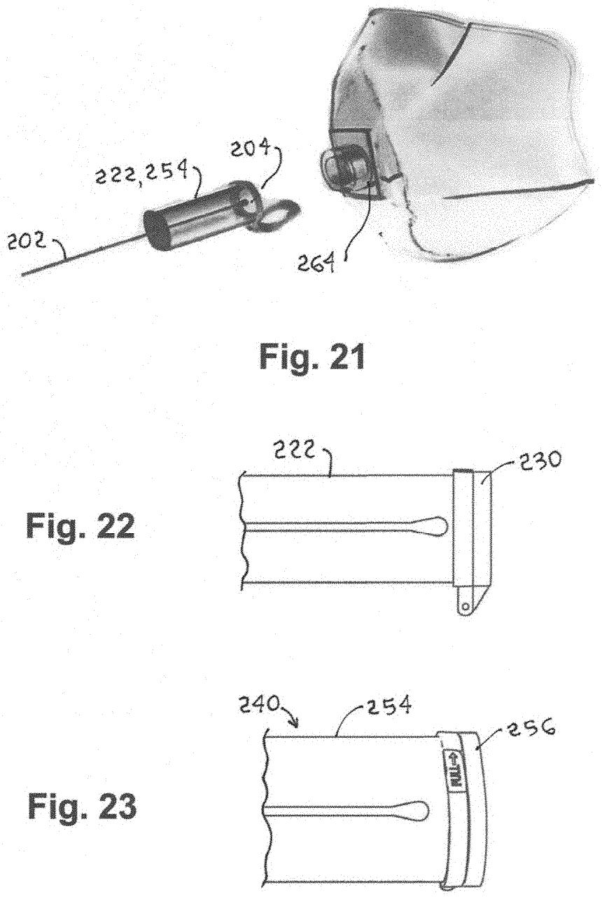

After the sample has been obtained, the collection swab 202 is removed from the swab access port 264 and the swab tip 204 is withdrawn into the tubular chambers 222, 254, as shown in FIGS. 20 and 21. The chamber cap 230 is closed onto the tubular chamber 222, as shown in FIG. 22. If the collector swab module 240 is used, the chamber cap 256 is closed onto the tubular chamber 254, as shown in FIG. 23. The tubular grip 212, 252 (not shown) may be replaced, as desired. Once in the closed state, shown in FIGS. 22 and 23, the tubular chamber 222, 256 can be transported for biological reaction testing, or interim storage, without further exposure to the ambient environment. It can be appreciated by one skilled in the art that limited ambient environmental exposure of the collected sample on the swab tip 204 occurs only when the tubular chamber 222, 254 is being inserted into, and then being withdrawn from, the swab access port 264, while the respective chamber cap 130, 156 is open.

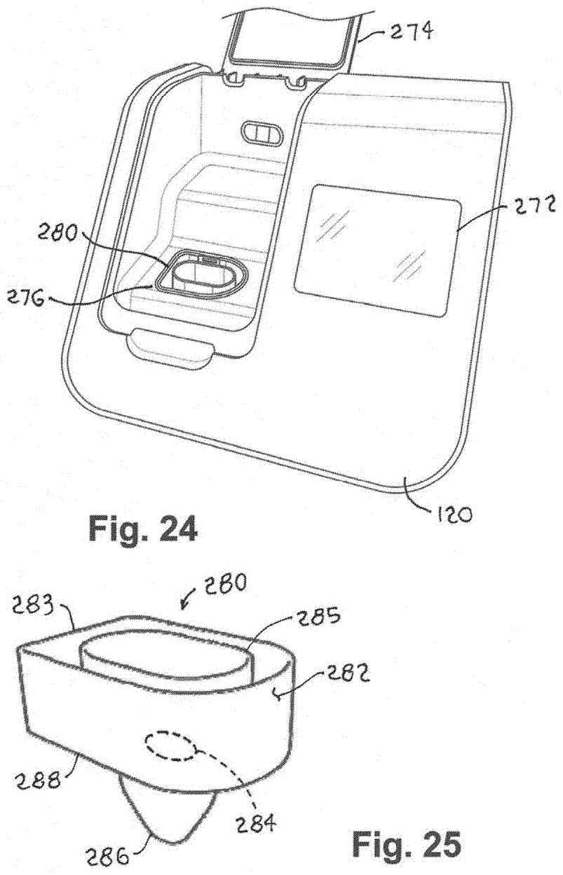

There is shown in FIG. 24 a detail view of the pathogen detection station 120, such as manufactured by Abbott Laboratories of Scarborough, Me., Other pathogen detection station 120, may be Antigen Test, RT-LAMP, LAMP, Serology RT-PCR, PCR which may be used for DNA/RNA testing and extraction for forensic, toxicology, pathology and the like. The pathogen detection station 120 includes a control screen 272, and a hinged cover 274 which can be opened to expose a pathogen test station 276. In the test setup shown, a testing basin 280 is provided in the pathogen test station 276. The testing basin 280, shown in FIG. 22, includes an oval wall 282 and a flat wall portion 283 on a perimeter of a planar base 284. The oval wall 282 and the flat wall portion 283 enclose a cylindrical channel 285. An elution buffer reservoir 286 depends from a reservoir opening 284 in the planar base 288. A fluid buffer or reactant, such as a viral transport medium, can be placed into the elution buffer reservoir 286 via the reservoir opening 284 enclosed by the cylindrical channel 184. The portable pathogen detection station 120 is battery powered and can be moved to a specified location as desired.

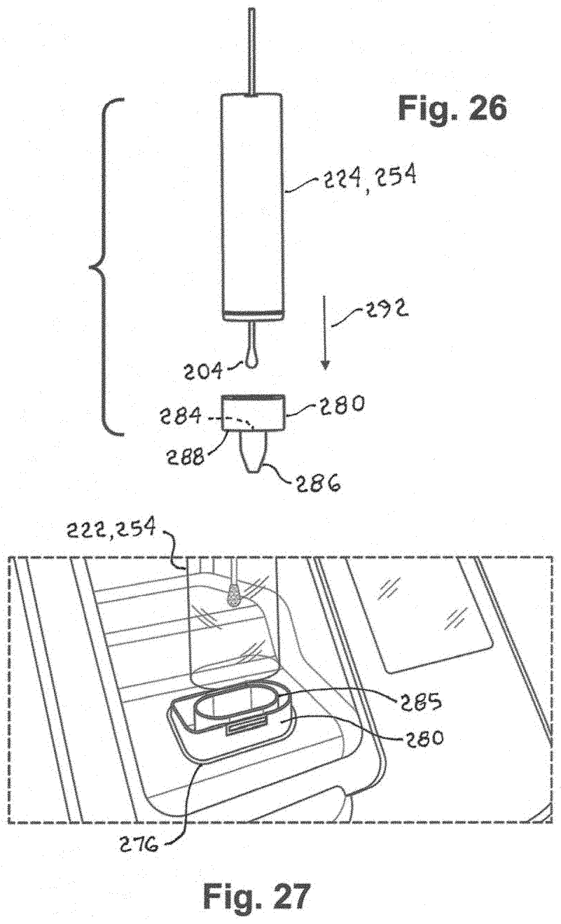

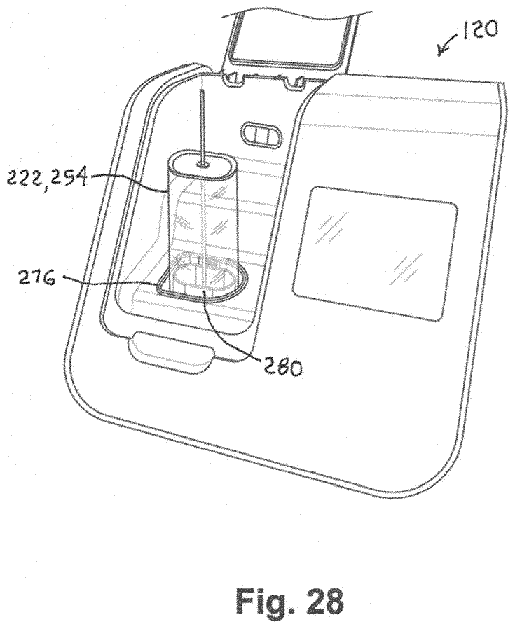

As shown in FIG. 26, the tubular chamber 222, 254 is configured for mating with the testing basin 280. With the chamber cap 230, 257 removed (not shown), the swab tip 204 is exposed and can be placed into the elution buffer reservoir 286 via the reservoir opening 284, as indicated by arrow 292. As seen from FIG. 27, the cross-sectional shape of the tubular chamber 222, 254 closely conforms to the cross-sectional shape of the cylindrical channel 285 such that the tubular chamber 222, 254 can be placed over the cylindrical channel 285 in an airtight fashion. Testing of a biological specimen is enabled by placement of the testing basin 280 into the pathogen test station 276, and the placement of the tubular chamber 222, 254 onto the testing basin 280, as shown in FIG. 28. When the tubular chamber 222, 254 is thus emplaced in the pathogen test station 276, the targeted, and possibly infectious, biological specimen is effectively encapsulated and contained.

The portability of the pathogen detection station 120 enables testing procedures in places remote from conventional medical facilities. In an exemplary embodiment, testing can be performed in an aircraft while in flight. It can be appreciated that an infected passenger may cause the spread of an airborne viral pathogen for a great distance. Employing the pathogen detection station 120 as part of standard on-board equipment can help to identify such an infected passenger, and steps can be taken to mitigate the spread of the disease.

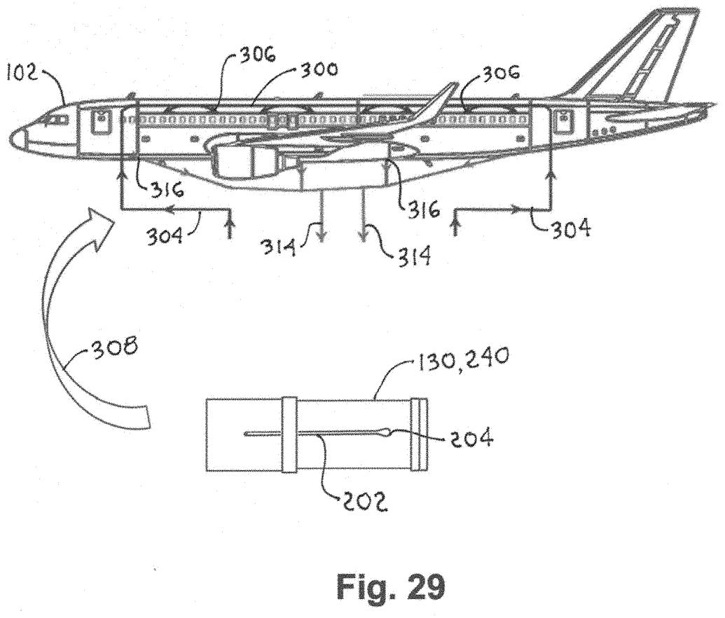

FIG. 29 is an exemplary illustration of the application of the collection swab module 130, 240 in the aircraft 102, or alternatively, in another commercial transportation vehicle or ground location (not shown). An aircraft ventilation system 300 in the aircraft 102 is used to control and purify ambient air for the benefit and comfort of passengers and crew. In the simplified illustration, the ventilation system 300 includes an air mixing unit (not shown) that brings conditioned air 304 into the passenger and crew compartments via input ducts 306. The input ducts 306 typically direct cabin air over the passengers' heads. Stale cabin air 314 is removed via exhaust ducts 316, typically located near the cabin floor. In an exemplary embodiment, at feast one collection swab module 130, 240 is emplaced, as indicated by arrow 308, so as to sample, using the collection swab 202, air flowing within the exhaust ducts 316. It can be appreciated that the air in the exhaust ducts 316 is placed into motion past the collection swab 202 by fan components in the aircraft ventilation system (not shown).

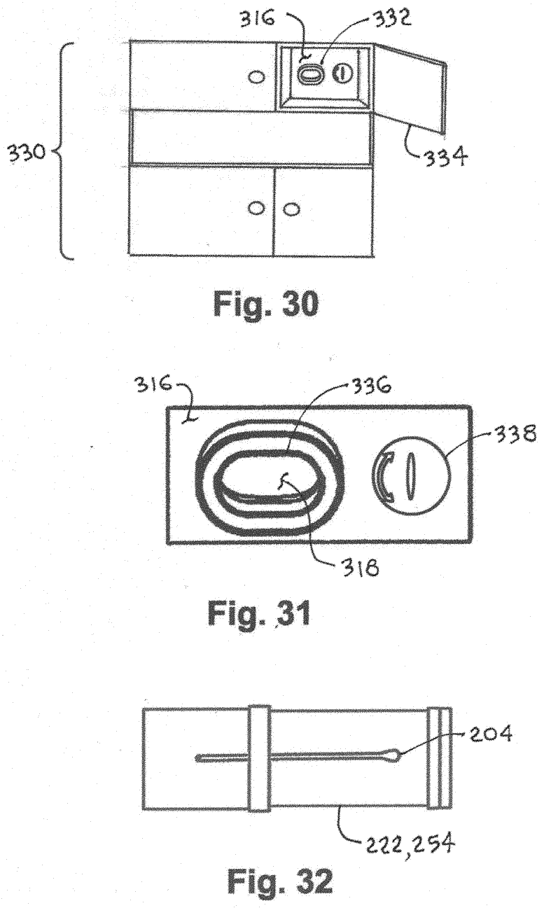

In an exemplary embodiment, shown in FIG. 30, a user, such as a flight crew member, can access an exhaust duct swab port 332 behind a swab port access door 334 in an aircraft galley panel 330, for example. As shown on FIG. 31, the exhaust duct swab port 332 has a substantially oval shape conforming to the shape of the swab module 130, 240, although other cross-sectional shapes can be used for the exhaust duct swab port 332 and the swab module 130, 240. An air passage 336 in the exhaust duct swab port 332 provides selective access from the cabin to the air stream in the exhaust duct 316.

This feature allows for a more efficiently sampling of possible airborne pathogens in the cabin air as the moving airflow in the exhaust duct 316 passes a greater volume of air over the collection swab 202 in a given time period than if the collection swab 202 were merely exposed to ambient cabin air. A control knob 338 is rotated to remove a blocking insert 318 from the air passage 336 after the swab module 130, 240 has been mated to the exhaust duct swab port 332. The swab tip 204 is then moved onto the airstream in the exhaust duct 316. The control knob 338 is also used to reposition the blocking insert 318 in air passage 336 before the swab module 130, 240 is to be removed from the exhaust duct swab port 332.

Placement of the tubular chamber 222, 254, shown in FIG. 32, over the exhaust duct swab port 332 provides an airtight fit so that the tubular chamber 222, 254 can remain in place when positioned onto the exhaust duct swab port 332. The tubular grip 212, 252 can be removed to expose the swab applicator shaft 206. The flight crew member can then operate the control knob 338 to open the exhaust duct swab port 332, and position the swab tip 204 in the airstream inside the exhaust duct 306 for pathogen detection.

Once a predetermined test period has expired, the swab tip 204 can be withdrawn from the airstream, the tubular grip 212, 252 can be replaced, the control knob 338 can be used to close the exhaust duct swab port 332, the tubular chamber 222, 254 can be removed from the exhaust duct swab port 332, and the chamber cap 230, 256 can be closed for stowage or transport. At a predetermined time, the collection swab module 130, 240 can be uncapped and placed into the testing basin 280 of the pathogen detection station 276 for testing, as shown in FIG. 28.

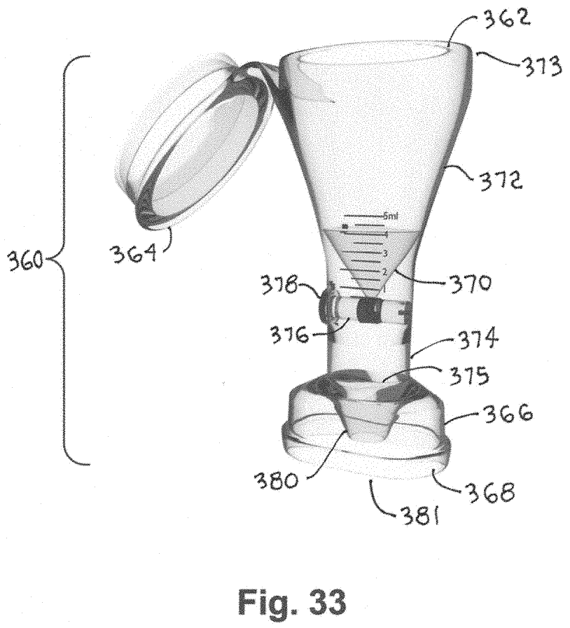

An alternative to the method of obtaining a sample by using the collection sample module 244 of FIG. 2, is using a fluid receptacle 360, shown in FIG. 33. The fluid receptacle 360 includes a tapered cylinder 362 with an attached receptacle cover 364 at an upper end 373 and an attached receptacle base 366 at a lower end 375. There is a conical reservoir 370 disposed midway in the tapered cylinder 362 where a flared section 372 transitions into a neck section 374. A fluid flow knob 378 is used to open and close a fluid delivery valve 376 that is disposed at the base of the conical reservoir 370. A fluid delivery funnel 380 is housed in the receptacle base 366, positioned so as to direct the flow of any fluid being dispensed from the conical reservoir 370. The receptacle base 366 provides a testing basin interface collar 382 that is configured to mate with the testing basin 280 of the pathogen test station 176, shown in FIG. 21.

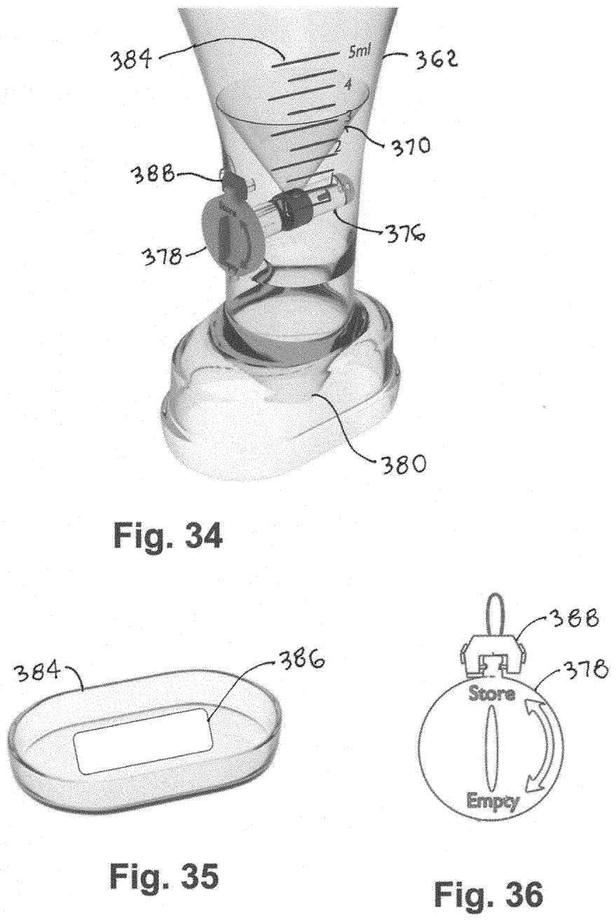

The fluid receptacle 60 may be used to collect fluid samples, such as saliva, from a test subject. A specified volume of fluid, indicated by graduation markings 384 on the tapered cylinder 362, is collected in the conical reservoir 370, shown in FIG. 34. A receptacle base cover 384, shown in FIG. 35, is configured to be frictionally secured to the testing basin interface collar 382. A liquid contact indicator 386, that changes color with an application of fluid, is secured to the receptacle base cover 384. As shown in FIGS. 34 and 36, the fluid flow knob 378 is prevented from rotating by a removable security tab 388. In a ‘store’ position, collected fluid is retained in the conical reservoir 370. In an empty position, a predetermined amount of fluid is allowed to flow through the fluid delivery funnel 380.

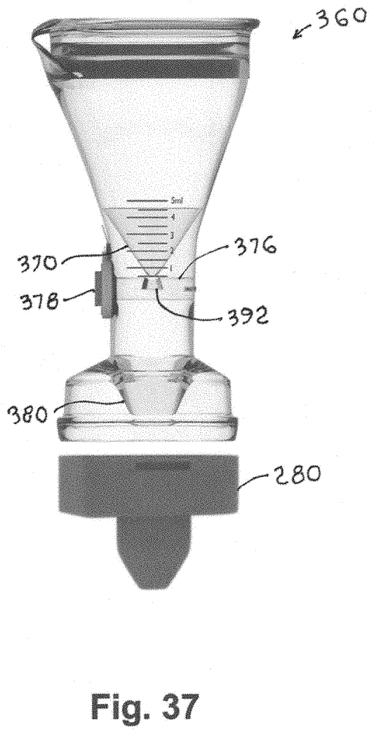

FIG. 37 illustrates the mating of the fluid receptacle 360 to the testing basin 280. The fluid delivery valve 376 includes a flow valve reservoir 392 that is configured to receive a predetermined amount of fluid from the conical reservoir 370 when the fluid flow knob 378 is in the ‘store’ position. When fluid flow knob 378 is turned to the ‘empty position, the predetermined amount of fluid is dispensed through the fluid delivery funnel 380 and into the testing basin 280.

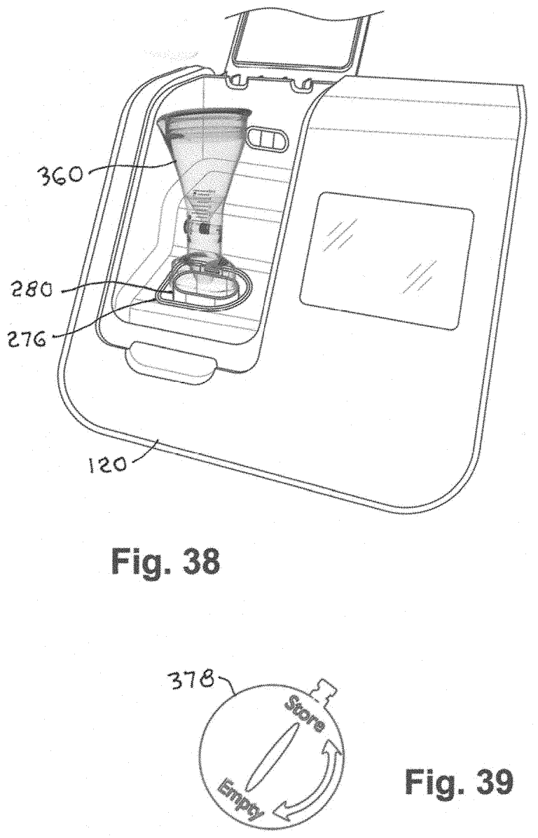

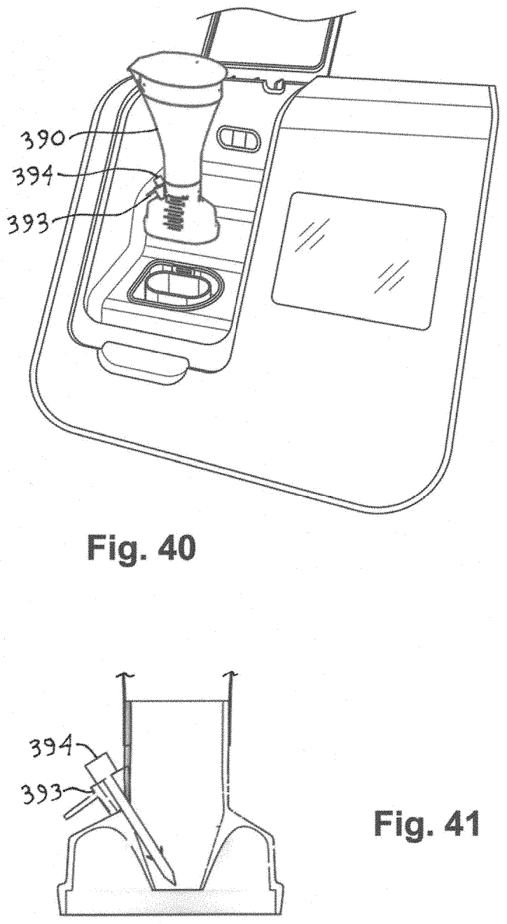

FIG. 38 shows the fluid receptacle 360 and the testing basin 280 properly emplaced in the pathogen test station 176 of the pathogen detection station 170. In this testing mode, the fluid flow knob 378 can be rotated to the ‘empty’ position, as shown in FIG. 39, to dispense the predetermined amount of fluid into the testing basin. In an alternative embodiment, shown in FIG. 40, a fluid receptacle 390 may include a puncture stopper 392 through which a syringe 394 may be inserted so as to dispense a fluid into the testing basin 280 (not shown), as illustrated in FIG. 41.

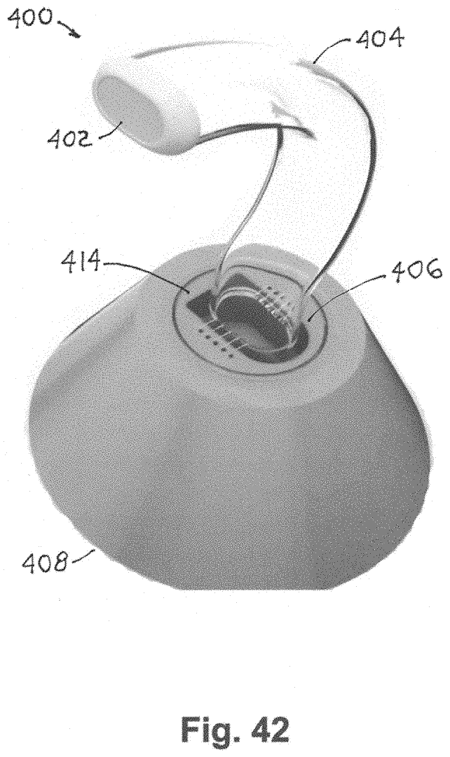

An alternative to the method of using the particulate matter cassette 248, shown in FIG. 27, to obtain samples of a virus or other pathogen filtered from ambient air, is by using the pathogen detection station 170 in conjunction with an air/breath collector 400, shown in FIG. 42. The air/breath collector 400 includes a gated mouth piece 402 covering one end of an air tube 404, the other end of the air tube 404 having an air tube flange 414 supported on an air collector base 408. The gated mouth piece 402 opens as a subject exhales into the air tube 404, whereby the exhaled air travels along the air tube 404, through an air intake manifold 406, and into the air collector base 408, as described in greater detail below.

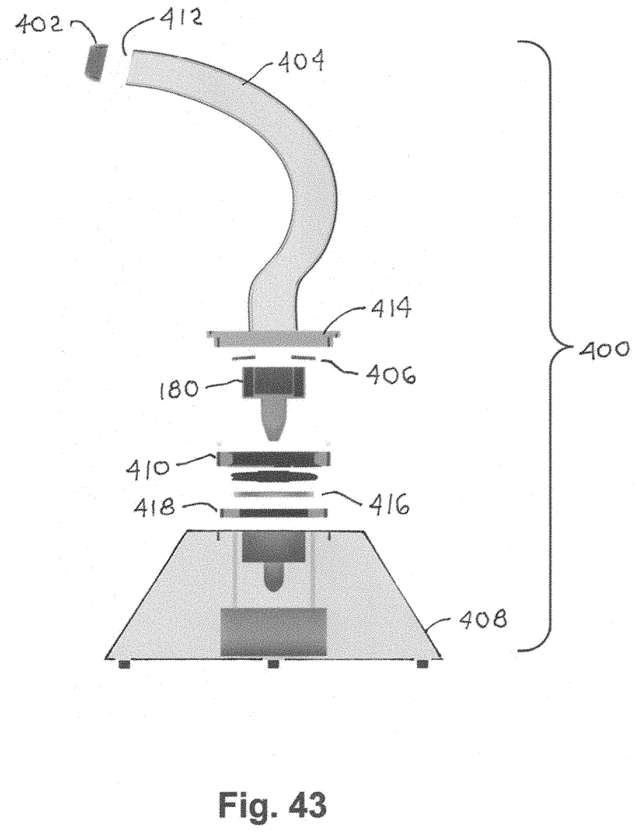

FIG. 43 is an exploded side view of the air/breath collector 400. A fan 410 in the air collector base 408 functions to draw air or the breath, exhaled by a subject, into the receiving end 412 of the air tube 404 via the gated mouth piece 402. The resulting air flow is diverted by the air intake manifold 406 so as to flow down the sides of the testing basin 280, through an ultra-low particulate air (ULPA) filter 416, through the ultraviolet light panel 418, and out the bottom of the air collector base 408. Note that the air intake manifold 406 is disposed between the air tube flange 414 and the testing basin 280. Moisture or air droplets in the airflow do not enter the air intake manifold but collect in the testing basin 280 to serve as samples in the process of testing for airborne pathogen.

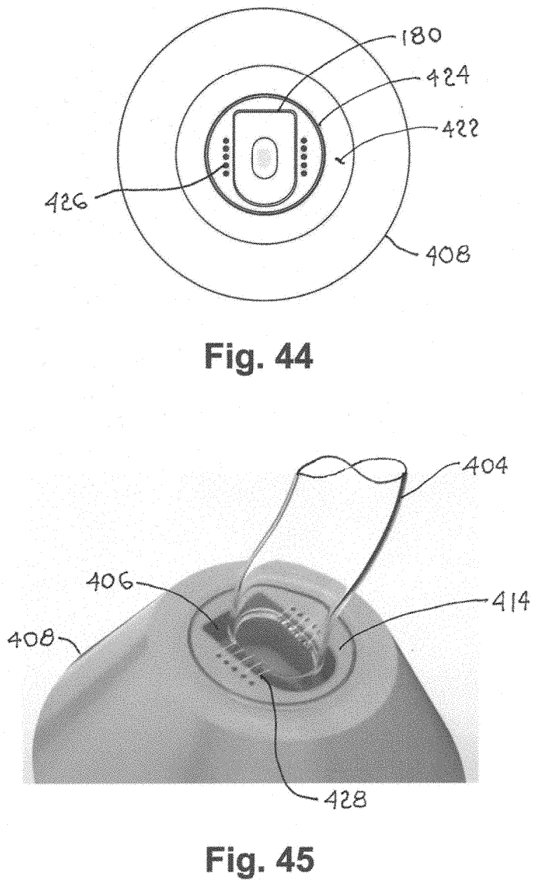

FIG. 44 presents a detail view of the upper surface 422 of the air collector base 408. A circular recess 424 is provided in the upper surface 422 to secure the air tube 404 (not shown) in place and eliminate air leakage between the air tube 404 and the air collector base 408. The circular recess 424 includes a plurality of small air flow vent holes 426 by which the airflow may be directed across the outer surface of the testing basin 280.

FIG. 45 shows the air tube 404 installed on the air collector base 408. The air intake manifold 406 includes a plurality of airflow microchannels 428 by which the airflow in the air tube 404 passes under the air tube flange 414, through the plurality of airflow microchannels 428, into the plurality of respective airflow vent holes 426, and into the air collector base 408.

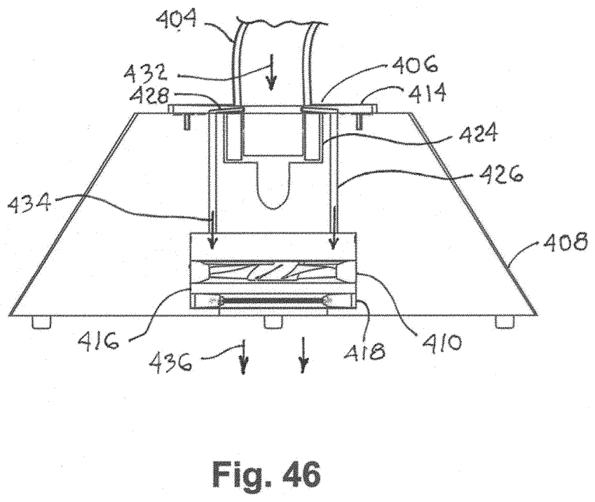

FIG. 46 shows a cross sectional view of the air tube 404 mounted onto the air co lector base 408. As described above, the air intake manifold 406 is disposed between the air tube flange 414 and the testing basin 280. The testing basin 280 is seated in the circular recess 424. A specially designed version of the test basin 280 may accommodate a nasal, breath, saliva, saline or mouth rinse, or blood sample for example. A fan 410 is provided at the lower end of the base vent channel 430, Incoming airflow, indicated by arrow 432, flows through the air tube 404, through the airflow microchannels 428, through the airflow vent holes 426, as indicated by arrows 434, and are exhausted from the air collector base 408, as indicate by arrows 436. The airflow is directed through the ULPA filter 416 and an ultraviolet light panel 418 after passing through the air intake manifold 406. The ultraviolet light panel 418 functions to kill pathogens in the air flow before being exhausted from the air collector base 408.

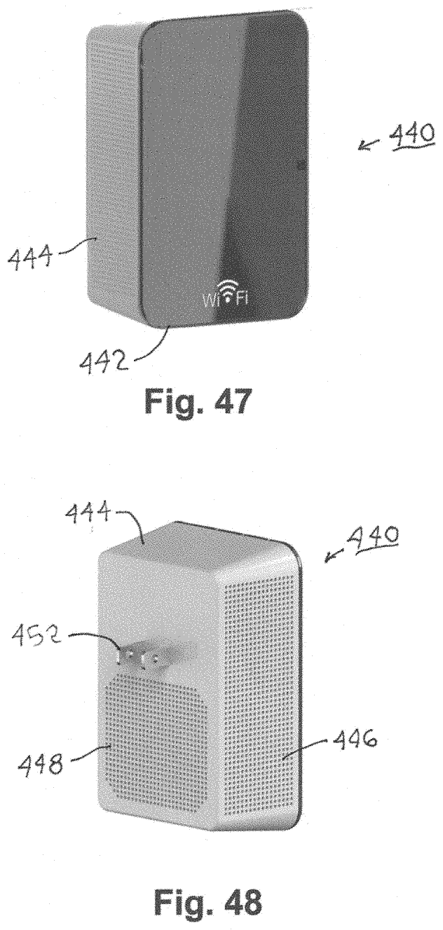

In an alternative embodiment, physical matter collected for the physical specimen module 242 may be provided by collection swabs exposed to an ambient air stream by means of an ambient air swab module 440, shown in FIG. 47. The ambient air swab module 440 is a portable closed unit including a front panel 442 secured to a module housing 444 to define a contained volume. The module housing 444 includes side vents 446 and rear vents 448, with electrical prongs 452 used for mounting the ambient air swab module 440 to an electrical outlet (not shown) to provide electrical power, as shown in FIG. 48.

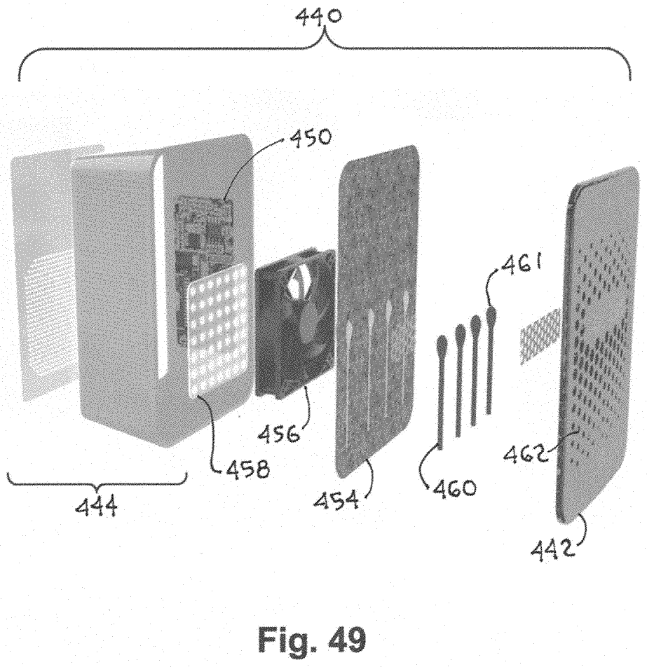

FIG. 49 is an exploded view of the ambient air swab module 440 showing the front panel 442 and the module housing 444. A swab support panel 454 is disposed adjacent the front panel 442. In the example provided, the swab support panel 454 is configured to hold up to four collection swabs 460 in an airstream directed at the collection swabs 460 from front panel vents 462 in the front panel 442. A custom swab support panel 460 can be designed to collect/accommodate a nasal, breath, saliva, saline or mouth rinse, or blood sample for example. a collection swab may include any one of: (i) substrate, (ii) cassette, (iii) custom swab or specimen retention holder, (iv) antigen test cassette, (v) air or breath sampling or retention vessel, (vi) and/or a pathogen/drug test strip/cassette/assay, (vii) condenser. The swab tips 461 are formed from synthetic fibers frayed at, the ends for optimal collection of particles in the air stream. A custom swab tips 461 may contain a collection swab may include any one of: (I) substrate, cassette, (iii) custom swab or specimen retention holder, (iv) antigen test cassette, (v) air or breath sampling or retention vessel, (vi) and/or a pathogen/drug test strip/cassette/assay, (vii) condenser. A fan 456 is mounted adjacent to, and behind, the swab support panel 454 to draw in air through the front panel vents 462. An ultraviolet light panel 458 is mounted behind the fan 456 to kill any pathogens present in the airflow after passing over the swab support panel 454 and returning to the cabin air. A control board 450 may be secured to the inside of the module housing 444. After the collection swabs 460 have been exposed to ambient air for a predetermined amount of time, the collection swabs 460 can be removed and placed in the physical specimen module 242 for evaluation at a testing device or the disease detection docking station 230, as shown in FIG. 27.

It is to be understood that the description herein is only exemplary of the invention, and is intended to provide an overview for the understanding of the nature and character of the disclosed autonomous aircraft disease detection system. The accompanying drawings are included to provide a further understanding of various features and embodiments of the method and devices of the invention which, together with their description serve to explain the principles and operation of the invention.

What is claimed is:

1. An autonomous aircraft disease detection system suitable for notifying a ground station of the presence of a viral pathogen on board an in-flight aircraft, said system comprising:
a medical device on board the aircraft for obtaining at least one in-flight medical sample;
a pathogen detection station having a pathogen test station and a disease sample port for receiving said at least one medical sample, said pathogen detection station producing medical data and readings upon analyzing said at least one medical sample in said pathogen test station or in said disease sample port;
a disease detection docking station in communication with said pathogen detection station, said disease detection docking station functioning to convert said medical data and readings into disease detection results;
a disease detection data processor in communication with said disease detection docking station via a disease detection data link, said disease detection data processor functioning to convert said disease detection results into computer-readable instructions and digital medical data;
a first transmitter activation device in communication with said disease detection data processor via a first transmitter activation signal link, said first transmitter activation device responsive to said computer readable instructions when said digital medical data includes an indication of a health emergency or an indication of an viral pathogen;
a first transmitter connected to said first transmitter activation device via a first transmission data link, said first transmitter establishing a first satellite communication link with a first satellite to notify the ground station of said indication of a health emergency or said indication of a viral pathogen when activated by said first transmitter activation device;
a second transmitter activation device in communication with said disease detection data processor via a second transmitter activation signal link, said second transmitter activation device responsive to said computer readable instructions when said digital medical data includes said indication of a health emergency or said indication of a viral pathogen; and
a second transmitter connected to said second transmitter activation device via a second transmission data link, said second transmitter establishing a second satellite communication link with a second satellite to notify the ground station of said indication of a health emergency or said indication of an viral pathogen when activated by said second transmitter activation device and
a third transmitter connected to the aircraft wireless network to onboard devices over a wireless network to notify authorities or flight crew of a health emergency.

2. The system of claim 1 wherein said medical device comprises:
a collection swab having a swab applicator shaft and a swab tip, said swab tip formed from a plurality of synthetic fibers; and
a collection swab transport tube enclosing said collection swab, said collection swab transport tube having a chamber cover hingedly attached to a cover anchor band at a chamber end, said chamber end configured to mate with a testing basin sized and shaped to fit into a basin recess in said pathogen test station, said collection swab provided to said pathogen test station as said medical sample.

3. The system of claim 2 further comprising a plastic face shield configured to fit against the face of a subject being tested for the presence of a viral pathogen or other airborne substance, said face shield including an access opening in a swab access port, said access opening sized and shaped to accommodate said chamber end so as to enable insertion of said collection swab into a mouth or a nasal cavity of said subject.

4. The system of claim 1 further comprising an exhaust duct swab test station having an oval cylindrical swab port extending from an aircraft exhaust duct, said oval cylindrical swab port configured to mate with said chamber end, said oval cylindrical swab port further including an air passage providing access to exhaust air flow in said aircraft exhaust duct, said exhaust duct swab test station further including a control knob for selectively opening and closing said air passage.

5. The system of claim 1 wherein said medical device comprises:
a tapered cylinder with a collector cover at a receiving end of said tapered cylinder;
a dispensing end of said tapered cylinder shaped as a hollow cone;
a conical reservoir secured in said tapered cylinder between said collector cover and said hollow cone, said conical reservoir positioned against a fluid delivery valve secured in said tapered cylinder, said fluid delivery valve operative to selectively allow a fluid specimen in said conical reservoir to pass into said hollow cone;
a dome base at said dispensing end, said dome base having, an interface collar at a dome base opening, said dome base enclosing said hollow cone; and
a testing basin sized and shaped to fit into a basin recess in said pathogen test station, said interface collar being sized and shaped to mate with said testing basin such that said fluid specimen passes through said hollow cone and collects in said testing basin to be provided to said pathogen test station as said medical sample.

6. The system of claim 1 wherein said medical device comprises:
an air tube having, at a first end, an air tube flange supported on an air intake base;
said air tube having, at a second end, a gated mouth piece functioning to enable the collection of fluid specimen particles in a breath sample taken from a subject at said gated mouth piece;
a testing basin secured in a base basin recess in said air intake base adjacent said air tube flange;
a fan disposed in said air intake base; and
an air intake manifold, said air intake manifold disposed between said air tube flange and said fan such that operation of said fan draws said breath sample from said subject into said gated mouth piece, through said air tube, and through said air tube flange:
wherein said air intake manifold functions to divert the flow of said breath sample into said air intake base while allowing said fluid specimen particles in said breath sample to collect in said testing basin, said fluid specimen particles collected in said testing basin provided to said pathogen test station as said medical sample.

7. The system of claim 1 wherein said medical device comprises:
a swab support panel holding at least one collection swab, said at least one collection swab having a swab applicator shaft and a swab tip, said swab tip formed from a plurality of synthetic fibers;
said swab support panel enclosed in an ambient air swab module housing, said ambient air swab housing having a mounting case and a front panel, said front panel including a plurality of front panel vents;
said ambient air swab housing further enclosing a fan, said fan functioning to intake ambient air through said front panel vents so as to flow said ambient air over said at least one swab tip on said swab support panel; and
an ultraviolet light panel mounted adjacent said swab support panel such that said fan flows said ambient air over said ultraviolet light panel after flowing said ambient air over said swab support panel, whereby said ultraviolet light panel functions to irradiate airborne viral pathogens.

8. The system of claim 1 wherein at least one of said first transmitter and said second transmitter continually transmits the current GPS location of the aircraft, so as to track the migration of a possibly infectious onboard disease to the location of the next landing destination of the aircraft.

9. The system of claim 1 wherein said first transmitter functions as an aircraft emergency location transmitter (ELT) and said second transmitter functions as part of an autonomous distress tracking (ADT) system, said first and second transmitters further functioning to broadcast distinctive signals and data on designated frequencies conforming to at least one of emergency location transmitter or emergency location beacon standards defined by at least one of: (i) the International Civil Aviation Organization (ICAO) for Global Aeronautical Distress Safety System (GAUSS), (ii) the International Maritime Organization (IMO) for Global Maritime Distress Safety System (GMDSS), (iii) 911 Services, or (iv) Emergency Services.

10. An autonomous aircraft disease detection system suitable for notifying a ground station of the presence of a viral pathogen on board an in-flight aircraft, said system comprising:
a pathogen specimen collector;
a pathogen detection station having a pathogen test station and a disease sample port for receiving at least one pathogen test specimen, said pathogen detection station producing medical data and readings upon analyzing said at least one pathogen test specimen in said pathogen test station or in said disease sample port;
a disease detection docking station with a disease sample port, said disease detection docking station in communication with said pathogen detection station, said disease detection docking station functioning to convert said medical data and readings from said pathogen detection station and medical data and readings from said disease sample port into disease detection results;
a disease detection data processor in communication with said disease detection docking station via a disease detection data link, said disease detection data processor functioning to convert said disease detection results into computer-readable instructions and digital medical data;
a transmitter activation device in communication with said disease detection data processor via a transmitter activation signal link, said transmitter activation device responsive to said computer readable instructions when said digital medical data includes an indication of a health emergency or an indication of a viral pathogen; and
a transmitter connected to said transmitter activation device via a transmission data link, said transmitter establishing a satellite communication link with a satellite to notify the ground station of said indication of a health emergency or said indication of a viral pathogen when activated by said transmitter activation device.

11. The system of claim 10 wherein said pathogen specimen collector comprises:
a tapered cylinder with a collector cover at a receiving end of said tapered cylinder;
a dispensing end of said tapered cylinder shaped as a hollow cone; and
a conical reservoir secured in said tapered cylinder between said collector cover and said hollow cone, said conical reservoir positioned against a fluid delivery valve secured in said tapered cylinder, said fluid delivery valve including a valve reservoir, said fluid delivery valve operative to selectively allow a fluid specimen in said conical reservoir to be transferred, via said valve reservoir, into said hollow cone; and
a dome base at said dispensing end, said dome base having an interface collar at a dome base opening, said dome base enclosing said hollow cone.

12. The system of claim 11 further comprising a testing basin secured in a basin recess in said pathogen test station, wherein said interface collar of said dome base is sized and shaped to mate with said testing basin such that said testing basin is aligned to receive said fluid specimen from said hollow cone when said interface collar is placed onto said testing basin.

13. The system of claim 12 wherein said testing basin comprises a planar base with an oval wall and a flat wall portion along a perimeter of said planar base so as to form a fluid specimen receptacle, said oval wall with a flat wall portion further enclosing a cylindrical channel providing a reservoir opening in said planar base, said planar base having an elution buffer reservoir depending from said reservoir opening.

14. The system of claim 10 wherein said pathogen specimen collector comprises:
an air tube having, at a first end, an air tube flange supported on an air intake base;
said air tube having, at a second end, a gated mouth piece functioning to enable the collection of fluid specimen particles in a breath sample taken from a subject at said gated mouth piece; and
a testing basin secured in a base basin recess in said air intake base adjacent said air tube flange.

15. The system of claim 14 wherein said pathogen specimen collector further comprises:
a fan disposed in said air intake base; and
an air intake manifold, said air intake manifold disposed between said air tube flange and said fan such that operation of said fan draws said breath sample from said subject into said gated mouth piece, through said air tube, and through said air tube flange;
wherein said air intake manifold functions to divert the flow of said breath sample into said air intake base while allowing said fluid specimen particles in said breath sample to collect in said testing basin, said testing basin functioning as a fluid specimen receptacle for said breath sample.

16. The system of claim 15 wherein said air intake base further comprises an ultraviolet light panel mounted adjacent said fan such that said fan flows said breath sample from said air intake manifold over said ultraviolet light panel, whereby said ultraviolet light panel functions to irradiate airborne viral pathogens.

17. The system of claim 10 wherein said disease detection docking station functions to obtain said medical data and readings from one or more of a physical specimen, a collection sample, a digital data storage device, and particulate matter analyzed at said disease sample port.

18. An autonomous aircraft disease detection system suitable for notifying a ground station of the presence of a viral pathogen on board an in-flight aircraft, said system comprising:
a testing basin having a planar base with an oval wall and a flat wall portion along a perimeter of said planar base so as to form a fluid specimen receptacle, said oval wall with said flat wall portion further enclosing a cylindrical channel providing a reservoir opening in said planar base, said planar base having an elution buffer reservoir depending from said reservoir opening;
a collection swab having a swab applicator shaft and a swab tip;
a pathogen detection station having a pathogen test station with a basin recess, said basin recess sized and shaped to receive said testing basin, said pathogen detection station producing medical data and readings upon analyzing a fluid specimen placed into said testing basin;
a disease detection docking station in communication with said pathogen detection station, said disease detection docking station functioning to convert said medical data and readings into disease detection results;
a disease detection data processor in communication with said disease detection docking station via a disease detection data link, said disease detection data processor functioning to convert said disease detection results into computer-readable instructions and digital medical data;
a transmitter activation device in communication with said disease detection data processor via a transmitter activation signal link, said transmitter activation device responsive to said computer readable instructions when said digital medical data includes an indication of a health emergency or an indication of a viral pathogen; and
a transmitter connected to said transmitter activation device via a transmission data link, said transmitter establishing a satellite communication link with a satellite to notify the ground station of said indication of a health emergency or said indication of a viral pathogen when activated by said transmitter activation device.

19. The system of claim 18 wherein said collection swab is housed in a collection swab transport tube, said collection swab transport tube including a tubular grip removably attached to a tubular chamber, said tubular chamber being a hollow cylindrical component having a chamber seal secured to a first chamber end and a chamber cap covering a second chamber end, said second chamber end configured to mate with said testing basin.

20. The system of claim 18 wherein said system further comprises a swab support panel removably securing at least one said collection swab, said swab support panel enclosed in an ambient air swab module, said ambient air swab module having a front panel with front panel vents, a fan, and an ultraviolet light panel, said fan functioning to intake ambient air through said front panel vents so as to flow said ambient air over said at least one collection swab on said swab support panel, said ultraviolet light panel mounted adjacent said swab support panel such that said fan flows ambient air over said ultraviolet light panel after passing over said swab support panel, whereby said ultraviolet light panel functions to irradiate airborne viral pathogens.
